(19) United States
(12) Patent Application Publication
(10) Pub. No.: US 2004/0131589 A1
(43) Pub. Date: Jul. 8, 2004

(54) Materials and methods for inhibition of IgE production

(76) Inventors: Howard M. Johnson, Gainesville, FL (US); Mustafa G. Mujtaba, Bridgewater, MA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/667,133

(22) Filed: Sep. 19, 2003

Related U.S. Application Data
(63) Continuation of application No. 09/648,864, filed on Aug. 25, 2000.
(60) Provisional application No. 60/151,026, filed on Aug. 27, 1999.

(51) Int. Cl. A61K 38/21

(52) U.S. Cl. 424/85.4

(57) ABSTRACT

The subject invention concerns novel methods and materials for treating patients afflicted with allergic conditions, such as allergic rhinitis, atopic dermatitis, bronchial asthma and food allergy. The method of the subject invention comprises administering interferon tau (IFNτ) or a chimeric IFN (ovine IFNτ (1-27)/human IFNαD (28-166)) to a person afflicted with an allergic condition. When administered, INFτ and chimeric IFN suppress the production of IgE antibodies without toxic side effects. The subject invention also concerns chimeric ovine/human IFNs which can be used in the methods of the invention.

### CROSS-REFERENCE TO A RELATED APPLICATIONS

This application is a continuation of U.S. application Ser. No. 09/648,864, filed Aug. 25, 2000, which claims the benefit of U.S. Provisional Application No. 60/151,026, filed Aug. 27, 1999.

The subject invention was made with government support under a research project supported by National Institute of Health Grant No. CA69959 and R37AI25904. The government has certain rights in this invention.

### BACKGROUND OF THE INVENTION

The interferons have been classified into two distinct groups: type I interferons, including IFNα, IFNβ, and IFNω (also known as IFNαII); and type II interferons, represented by IFNγ (reviewed by DeMaeyer et al., 1998). In humans, it is estimated that there are at least 17 IFNα non-allelic genes, at least about 2 or 3 IFNβ non-allelic genes, and a single IFNγ gene.

IFNα's have been shown to inhibit various types of cellular proliferation. IFNα's are especially useful against hematologic malignancies such as hairy-cell leukemia (Quesada et al., 1984). Further, these proteins have also shown activity against multiple myeloma, chronic lymphocytic leukemia, low-grade lymphoma, Kaposi's sarcoma, chronic myelogenous leukemia, renal-cell carcinoma, urinary bladder tumors and ovarian cancers (Bonnem et al., 1984; Oldham, 1985). The role of interferons and interferon receptors in the pathogenesis of certain autoimmune and inflammatory diseases has also been investigated (Benoit et al., 1993).

IFNα's are also useful against various types of viral infections (Finter et al., 1991). Alpha interferons have shown activity against human papillomavirus infection, Hepatitis B, and Hepatitis C infections (Finter et al., 1991; Kashima et al., 1988; Dusheiko et al., 1986; Davis et al., 1989). In addition, studies with IFNα and IFNγ have shown suppression of IgE production in allergic diseases (Noh et al., 1998; Hofstra et al., 1998; Lack et al., 1996; Dolen et al., 1995; Kimata et al., 1995; Gruschwitz et al., 1993).

Significantly, however, the usefulness of IFNα's has been limited by their toxicity: use of interferons in the treatment of cancer and viral disease has resulted in serious side effects, such as fever, chills, anorexia, weight loss, and fatigue (Pontzer et al., 1991; Oldham, 1985). These side effects often require (i) the interferon dosage to be reduced to levels that limit the effectiveness of treatment, or (ii) the removal of the patient from treatment. Such toxicity has reduced the usefulness of these potent antiviral and antiproliferative proteins in the treatment of debilitating human and animal diseases.

Interferon-tau (IFNτ) is a member of the type I IFN family but, unlike IFNα and IFNβ, IFNτ lacks toxicity at high concentrations in vitro and when used in vivo in animal studies (Bazer et al., 1989; Pontzer et al., 1991; Soos, Johnson, 1995; Soos, et al., 1995; Soos et al., 1997; Khan et al., 1998). IFNτ0 was originally identified as a pregnancy recognition hormone produced by trophoblasts cells of the placenta of ruminants such as sheep and cows (Bazer et al., 1991; Godkin et al., 1982; Imakawa et al., 1987; Johnson et al., 1994). It has been reported that a human IFNτ exists (Whaley et al., 1994) but this observation has not been confirmed. Thus, it is currently unknown as to whether there is a human IFNτ. INFτ exhibits antiviral and cell inhibitory properties which are very similar to that of IFNα and IFNβ (Bazer et al., 1989; Pontzer et al., 1991; Soos, Johnson, 1995). However, IFNτ lacks the cellular toxicity associated with high concentrations of IFNα and IFNβ (Bazer et al., 1989; Pontzer et al., 1991). Further, the weight loss and bone marrow suppression that is associated with administering high doses of IFNα and IFNβ to individuals is absent with IFNτ in animal systems (Soos, Johnson, 1995; Soos et al., 1995; Soos et al., 1997). Studies have shown that the N-terminus of type I IFNs play a role in the toxicity or lack thereof for an IFN (Pontzer et al., 1994; Subramaniam et al., 1995).

It has been reported that IFNτ suppresses the humoral and cellular responses in experimental allergic encephalomyelitis (EAE), an animal model for the autoimmune disease, multiple sclerosis (Mujtaba et al., 1998). It has been shown that INFτ suppresses the responses of lymphocytes to mitogens such as Con A and superantigens such as SEA and SEB (Soos, Johnson, 1995; Soos et al., 1995; Khan et al., 1998). There are also reports, again in the EAE model, that INFτ and other type I IFNs can induce IL-10 and TGFβ, but not IL-4, production by cells that have already been activated by antigen presenting cells (Soos, Subramaniam et al., 1995; Mujtaba et al., 1998; Mujtaba et al., 1997). INFτ has also been suggested for use in the treatment of Multiple Sclerosis in humans.

Production of IgE immunoglobulin is important in mediating allergic diseases such as allergic rhinitis, atopic dermatitis, bronchial asthma, and food allergy. Allergic sensitization of mice by intraperitoneal (ip) injection with ovalbumin (OVA) as an allergen and aluminum hydroxide as an adjuvant is a well characterized method of stimulating IgE production in vivo (Mancino et al., 1980; Miguel et al., 1977; Beck et al., 1989). When OVA-immunized mice are challenged with aerosolized OVA, they show inflammatory cell infiltration in the submucosal layer of the lungs (Kay et al., 1992; Hamelmann et al., 1996; Hamelmann et al., 1997). IgE can stimulate the release of certain chemotactic mediators from mast cells that can lead to active accumulation of macrophages and granulocytes at the site. Also, production of a further set of inflammatory molecules by these cells can lead to allergic asthma. Thus, allergen specific IgE production by B cells is important in the pathogenesis of allergic diseases.

Conventional therapy for allergic disease consists of decongestants and anti- histamines, which function to reduce symptoms after allergic responses occur. Thus, there remains a need in the art for a treatment of allergic diseases which lacks toxic side effects and functions to block allergic response, thus acting prior to symptomology.

### BRIEF SUMMARY OF THE INVENTION

The subject invention concerns novel methods and materials for treating patients afflicted with allergic conditions, such as allergic rhinitis, atopic dermatitis, bronchial asthma and food allergy. The method of the subject invention comprises administering a type I interferon, such as interferon tau (IFNτ), or a chimeric IFN (for example, ovine IFNτ (1-27)/human IFNαD (28-166)) to a person afflicted with an allergic condition. When administered, the interferon suppresses the production of allergen-specific IgE antibodies without toxic side effects. The subject invention also concerns chimeric ovine/human IFNs which can be used in the methods of the invention.

### BRIEF DESCRIPTION OF THE DRAWINGS

FIGS. 1A and 1B show the inhibition of OVA-specific IgE antibody production in OVA-sensitized mice by IFNτ treatment. BALB/C mice were immunized by ip injection with ovalbumin (OVA) mixed with aluminum hydroxide and boosted seven days later. The mice were exposed to aerosolized OVA (1% w/v) on days 19 and 20 after immunization for 20 minutes. Mice were treated daily with ip injections of IFNτ (5×10⁵ U/day) or PBS starting three days prior to immunization. Blood was collected 24 h prior to (FIG. 1A) and 24 h after (FIG. 1B) aerosolized OVA exposure. Direct ELISA was performed to detect OVA-specific IgE levels. Two to three mice per group were used, and average absorbance is shown. Control absorbance using a normal mouse serum has been subtracted out from each dilution point. Statistical significance for the inhibition of OVA-specific IgE antibody production was shown by Student's t test at all dilutions (except for the 10,000 dilution) for IFNτ treatment as compared to PBS treatment (p<0.05).

FIGS. 2A-2C show the histological evaluation of OVA-immunized mice after treatment with PBS or IFNτ. BALB/C mice were immunized with OVA and exposed to aerosolized OVA on day 20 after immunization and treated with PBS or IFNτ as previously described. Twenty-four hours after aerosolized OVA treatment, lungs from non-immunized (FIG. 2A), PBS treated (FIG. 2B), and IFNτ treated (FIG. 2C) mice were extracted, fixed, embedded in paraffin, sectioned and stained with hematoxylin and eosin for inflammatory cells. Arrows indicated the epithelium of the bronchiole.

FIG. 3 shows IL-4 levels in sera of OVA-immunized mice treated with PBS or IFNτ. BALB/C mice were immunized with OVA and exposed to aerosolized OVA and treated with PBS or INFτ as previously described in FIG. 1 description. Blood was collected 24 h after aerosolized OVA exposure, and a sandwich ELISA for IL-4 was performed. Two to three mice per group were used, and average amount (ng) of IL-4 is shown. Control level from naive mouse serum was subtracted from the PBS and IFNτ levels.

FIGS. 4A and 4B show in vivo treatment of OVA-immunized mice with IFNτ reduces OVA-stimulation of splenocytes. BALB/C mice were immunized with OVA and exposed to aerosolized OVA and treated with PBS or IFNτ as previously described in the FIG. 1 legend. Spleen cells (5×10⁵ cells/well) were cultured with OVA at 100 μg/ml for 72 h, after which the cultures were pulsed with tritiated thymidine. PBS-treated splenocytes were also incubated with BSA and MBP at 100 μg/ml (FIG. 4B). Cell associated radioactivity was quantified 12 h later using a β-scintillation counter, and data from one of three experiments are presented as mean cpm of quadruplicate wells±SD. Statistical significance for the inhibition of OVA-induced cell proliferation by IFNτ treatment as compared to PBS treatment was shown using Student's t test (p<0.001).

FIG. 5 shows in vitro treatment of splenocytes with type I IFNs inhibit OVA-specific proliferation. BALB/C mice were immunized by ip injection of OVA mixed with aluminum hydroxide and boosted 7 days later. The mice were exposed to aerosolized OVA (1% w/v) on day 19 and 20 for 20 minutes. Spleen cells (5×10⁵ cells/well) were cultured with 15000 U/ml of various IFNs and media in the presence or absence of 100 μg/ml OVA for 84 h, after which the cultures were pulsed with tritiated thymidine. Cell associated radioactivity was quantified 12 h later using a β-scintillation counter, and data from one of three experiments are presented as mean cpm of quadruplicate wells±SD. Inhibition of OVA specific splenocyte proliferation by all the IFNs was statistically significant as compared to OVA-specific splenocyte proliferation of OVA-sensitized medium-treated cells as shown by Student's t test (p<0.001).

FIGS. 6A-6C show immunoblot detection of mouse and human IgE in culture supernatants taken from ovalbumin (OVA)-sensitized mouse splenocytes or human myeloma B cells treated with various IFNs or media. FIG. 6A includes Lane 1, control mouse IgE; lane 2, RPMI 1640 supplemented with 10% FBS; lanes 3 and 4, 84 h supernatants from naive mouse splenocytes cultured in the absence or presence of OVA, respectively; lanes 5 and 6, 84 h supernatants from PBS-treated OVA-sensitized mouse splenocytes cultured in the absence or presence of OVA, respectively; lanes 7 and 8, 84 h supernatant from IFNτ-treated OVA-sensitized mouse splenocytes in absence or presence of OVA, respectively. FIG. 6B includes Lane 1, control mouse IgE; lane 2, RPMI 1640 medium only; lane 3, 84 h splenocyte culture in the absence of OVA; lanes 4, 5, 6, and 7, 84 h splenocyte (5×10⁵ cells/well) cultures with OVA in presence of media, IFNτ, IFNτ/IFNα chimeric, and IFNαD, respectively. FIG. 6C includes Lane 1, RPMI 1640 medium only; lanes 2, 3, 4, and 5, IgE producing U266BL cells, which were starved overnight, and incubated at 2×10⁵ cells/well for 96 h in the presence of media, 1.0×10⁴ U/ml IFNαD, IFNτ/IFNαD chimeric, and IFNτ, respectively.

FIG. 7 shows the inhibition of proliferation of the IgE-producing human myeloma B cell line U266 by type I IFNs. The IgE-producing U266BL cells, which were starved overnight, were incubated at 2×10⁵ cells/well in the presence of 1.0×10⁴ U/ml of IFNτ, IFNτ/IFNαD chimeric, IFNαD, and media 72 h. Cultures pulsed with tritiated thymidine, and cell associated radioactivity was quantified 12 h later using a β-scintillation counter, and data from one of three experiments are presented as mean cpm of quadruplicate wells±SD. Percent cell viability, as measured by trypan blue exclusion test, is presented above each bar. Statistical significance for the inhibition of cell proliferation was shown by Student's t test for all the IFN treatments as compared to the media treatment (p<0.001).

FIG. 8 shows the metabolic activity of human peripheral blood mononuclear cells (HPBMC) after treatment with IFNs. HPBMC were cultured in the presence of varying concentrations (250 to 100,000 U/ml) of IFNτ, IFNαD, and IFNτ/IFNαD chimeric for seven days. Metabolic activity of HPBMC was assessed by measuring cell proliferation and viability as described in the Materials and Methods and reported here as percent of the untreated control. Values for HPBMC treated with ovine IFNτ and IFNτ/IFNα chimeric were not significantly different from the untreated control whereas values for HPBMC treated with human IFNα were significantly different (p<0.05) from the untreated controls as determined using the Wilcoxon signed-rank test.

### DETAILED DESCRIPTION OF THE INVENTION

The subject invention concerns novel therapeutic and prophylactic methods for treating any condition where suppression or inhibition of IgE production is useful or beneficial, including allergic diseases and other IgE-related diseases or conditions. Disease conditions that can be treated according to the subject methods include, but are not limited to, allergic rhinitis, atopic dermatitis, bronchial asthma and food allergy. In the methods of the present invention, an effective amount of a composition comprising a type I IFN, such as IFNα, IFNβ, IFNτ or IFNω, or a chimeric IFN, is administered to a person having a condition where suppression or inhibition of IgE production is clinically desirable.

In one embodiment of the subject invention, an effective amount of IFNτ is administered to a person or animal afflicted with, or predisposed to, an allergic condition or other IgE-associated condition. The IFN used in the subject methods can be from any animal that produces the IFN, including but not limited to, primate, ovine, bovine and others.

In another embodiment of the subject invention, a mammalian IFN that has an amino acid sequence that provides the low toxicity of IFNτ with the bioactivity of other type I IFNs is used in the subject methods to treat a person or animal afflicted with, or predisposed to, an allergic condition or other conditions or diseases where suppression of IgE production or response is beneficial. In a preferred embodiment, an effective amount of a chimeric IFN comprising a mammalian IFNτ amino terminus and a human type I IFN carboxy terminus, such as that from IFNα, is administered to a person afflicted with, or predisposed to, an allergic condition or other IgE-associated condition. More preferably, the chimeric IFN protein comprises amino acid residues 1-27 of ovine IFNτ and amino acid residues 28-166 of human IFNα. In an exemplified embodiment, the IFNα is IFNαD.

The subject invention also concerns methods for suppressing IgE production and cell proliferation in vivo and in vitro using a type I interferon. As exemplified herein, splenocytes taken from an ovalbumin (OVA) immunized animal using either INFτ or a chimeric IFNτ protein suppressed OVA-induced proliferation and IgE production. Thus, the methods of the subject invention can be used to suppress IgE production in an animal or person. The methods of the subject invention can also be used to suppress IgE production in vitro.

The present invention also concerns methods for inhibiting B cell and T cell responses, including cell proliferation and cytokine production. As exemplified herein, type I IFNs can be used to inhibit production of IL-4. The cytokine IL-4 plays a central role in isotype switching of the B cells to IgE production.

Biologically active muteins (mutated proteins) of the subject polypeptides, as well as other molecules, such as fragments, peptides and variants, that possess substantially the same IgE-suppressive bioactivity as the subject IFN polypeptides, are contemplated within the scope of the subject methods. For example, IFNτ polypeptides that contain amino acid substitutions, insertions, or deletions that do not substantially decrease the biological activity and function of the mutant polypeptide in comparison to native polypeptide are within the scope of the present invention. Specifically contemplated within the scope of the invention are fragments of the type I IFN that retain substantially the same biological activity as the full length IFN. The muteins and fragments of IFNs can be readily produced using standard methods known in the art. For example, by using the Bal31 exonuclease (Wei et al., 1983), the skilled artisan can systematically remove nucleotides from either or both ends of the polynucleotide to generate a spectrum of polynucleotide fragments that when expressed provide the IFN fragment encoded by the polynucleotide.

Therapeutic application of the subject polypeptides and compositions containing them can be accomplished by any suitable therapeutic method and technique presently or prospectively known to those skilled in the art. The polypeptides can be administered by any suitable route known in the art including, for example, oral, parenteral, subcutaneous, or intravenous routes of administration. Administration of the polypeptides of the invention can be continuous or at distinct intervals as can be readily determined by a person skilled in the art.

The subject invention also concerns chimeric IFN polypeptides and the polynucleotides that encode them. In one embodiment, the chimeric IFNs comprise ovine and human IFN regions. Preferably, a chimeric IFN protein of the invention comprises an ovine IFNτ amino terminus and a human IFNα carboxy terminus. In an exemplified embodiment, the chimeric IFN protein comprises amino acid residues 1-27 of ovine IFNτ and residues 28-166 of human IFNαD. The polynucleotide sequences encoding the chimeric IFNs of the present invention can be readily constructed by those skilled in the art having the knowledge of the amino acid sequences of the subject polypeptides. As would be appreciated by one skilled in the art, a number of different polynucleotide sequences can be constructed due to the degeneracy of the genetic code. The choice of a particular nucleotide sequence could depend, for example, upon the codon usage of a particular expression system.

Compounds useful in the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive polypeptide is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art.

The compounds of the subject invention can also be administered utilizing liposome technology, slow release capsules, implantable pumps, and biodegradable containers. These delivery methods can, advantageously, provide a uniform dosage over an extended period of time.

Examples of carriers or diluents for use with the subject polypeptides include ethanol, dimethyl sulfoxide, glycerol, alumina, starch, and equivalent carriers and diluents. To provide for the administration of such dosages for the desired therapeutic treatment, new pharmaceutical compositions of the invention will advantageously comprise between about 0.1% and 45%, and especially, 1 and 15% by weight of the total of one or more of the polypeptides based on the weight of the total composition including carrier or diluent.

All patents, patent applications, provisional applications, and publications referred to or cited herein are incorporated by reference in their entirety to the extent they are not inconsistent with the explicit teachings of this specification.

### Materials and Methods

Interferons.

The ovine interferon tau (IFNτ) gene was expressed in Pichia pastoris using a synthetic gene construct (Heeke et al., 1996). IFNτ was secreted into the medium and was purified by successive DEAE-cellulose and hydroxylapatite chromatography to electrophoretic homogeneity as determined by SDS-PAGE and silver staining analysis. The purified protein had a specific activity of 2.9-4.4×10⁷ U/mg protein as measured by antiviral activity using a standard viral microplaque reduction assay on MDBK (Pontzer et al., 1991). The recombinant human IFNαD was from Biosource International, Camarillo, Calif. The “humanized” IFNτ/IFNαD chimeric protein was constructed using residues 1-27 of the ovine IFNτ and residues 28-166 of the human IFNαD and was expressed in Pichia pastoris as previously described for ovine IFNτ (Heeke et al., 1996).

IFNτ was administered intraperitoneally (ip) at 5×10⁵ U/mouse daily starting 96 h prior to immunization and continuing everyday thereafter for a month. Control mice received PBS.

Immunization of Mice.

BALB/C mice were immunized ip with 10 μg of ovalbumin (OVA) (Sigma, St. Louis, Mo.) precipitated with 5 mg aluminum hydroxide gel in a total volume of 100 μL. Aluminum hydroxide gel was prepared as previously described (Revoltella et al., 1969; Warner et al., 1968). Mice were immunized again 7 days after the initial immunization using the same protocol. The mice were exposed to aerosolized OVA from 1% OVA (w/v) in PBS on days 19 and 20 after immunization. Aerosolization was performed for 20 min using the Pari ls Jet+nebulizer and compressor (Pari Respiratory Equipment, Inc., Midlothian, Va.). Mice were housed and cared for at the Animal Resource Center (University of Florida), and all experimental animal uses were approved by the Institutional Animal Care and Use Committee (IACUC).

Histological Evaluation.

The lungs of OVA-immunized mice that had been exposed to aerosolized OVA were intratracheally perfused with 4% paraformaldehyde solution. The lungs were fixed for 2-3 days in the same solution after which lung samples were embedded in paraffin and sectioned. Samples were then stained with hematoxylin and eosin. Also, blood smears were prepared on slides from the same mice, and slides were stained with the “LEUKOSTAT” staining kit (Fisher Scientific, Pittsburgh, Pa.) for the determination of differential white blood cell count. A total of 150 white blood cells were evaluated.

Proliferation Assay.

Spleen cells taken from mice 21 days after immunization from PBS or IFNτ-treated mice were cultured at 5×10⁵ cell/well in presence of OVA for 72 to 84 h in RPMI 1640 medium containing 10% FBS. In other assays, PBS-treated mouse splenocytes were incubated at 5×10⁵ cells/well in presence of OVA and various IFNs (10,000 to 15,000 U/ml) for 72 to 84 h. The cultures were pulsed with [³H]-thymidine (1.0 uCi/well; Amersham, Indianapolis, Ind.) and harvested 12 h later on to filter paper discs using a cell harvester. Cell associated radioactivity was quantified using a β-scintillation counter and activity reported in CPM. Proliferation assays on the U266BL myeloma B cells were also carried out by incubating the cells in RPMI 1640 medium overnight prior to culturing 4×10⁵ cells/well with various IFNs at 10,000 to 15,000 U/ml in RPMI 1640 containing 4% FBS. Cultures were incubated for 72-84 h after which cells were pulsed with [³H]-thymidine prior to harvest 12 h later. Cell associated radioactivity was quantified using β-scintillation counter and activity reported in CPM. The U266BL cell line, an IgE producing myeloma that was isolated from the peripheral blood of a patient, provides a system to assess the direct effects of INFτ on an IgE producing cell (Nilsson et al., 1970). This cell line allows one to study the effects of INFτ on B cells with ongoing IgE synthesis.

Enzyme Linked Immunoadsorbent Assay.

OVA was resuspended in binding buffer (0.1 M carbonate/bicarbonate, pH 9.6) and absorbed onto the flat bottoms of plastic 96-well tissue culture wells overnight at 4° C. at a concentration of 2 μg/well and subsequently evaporated to dryness. The plates were treated with blocking buffer, 5% powdered milk in PBS, for 2 h in order to block nonspecific binding and then washed three times with PBS containing 0.05% Tween 20. Various dilutions of sera from BALB/C mice which were IFNτ-treated or PBS-treated or nonimmunized (naive) mice were added to the wells and incubated for 3 h at room temperature. After extensive washing, rabbit anti-mouse IgE antibody (Accurate, N.Y.) was added. Plates were washed three times prior to addition of 1:1000 diluation of horse radish peroxide (HRP) conjugated goat anti-rabbit immunoglobulin (Amersham Pharmacia Biotech, Piscataway N.J.). Color development was monitored at 490 ηm in an ELISA plate reader (BioRad, Richmond, Calif.) after the substrate solution (0.002M o-phenylenediamine dihydrochloride, 0.012% H₂O₂, 0.05 M Na Citrate, 0.05 M citrate) was added and the reaction terminated with 2M H₂SO₄.

For the detection of IL-4 in blood, sera samples were collected from PBS- or IFNτ treated mice and incubated in 96 well plates that had rabbit polyclonal anti-mouse IL-4 antibody (Biosource Int., Camarillo, Calif.) bound to it. After washing, 25 μg/ml of rat monoclonal anti-mouse IL-4 biotinylated antibody was added for 1 h incubation. A 1:1000 dilution of HRP-conjugated avidin was added after the incubation and washings, and substrate color development was monitored as described above. The limit of detection of the IL-4 ELISA was 7 ng/ml.

Western Blot.

Culture supernatants from both IFN or media treated OVA-sensitized splenocytes, and U266BL myeloma B-cells, were loaded at 10 ug/lane (total protein) on 15% and 10% SDS-PAGE READY GELS (BioRad, Richmond, Calif.) respectively, and run at 100 volts. Overnight transfer onto nitrocellulose membrane was carried out, after which the membrane was blocked with 5% milk in Tris-buffered saline pH 7.5, 0.1% Tween 20 for 1 h. Immunoblots were incubated with a 1:1000 dilution of either goat anti-human IgE (Biosource Int., Camarillo, Calif.) coupled to HRP or rabbit anti-mouse IgE (Accurate, N.Y.). The mouse IgE blot was incubated further after three washes with HRP conjugated anti-rabbit Ig (Amersham Pharmacia Biotech, Piscataway, N.Y.) for 1 h. Blots were washed and analyzed through film development.

Toxicity Assays of IFNs.

Toxicity assays using human peripheral blood mononuclear cells (HPBMC) were carried out by culturing HPBMC in the presence of varying concentrations of IFNτ, IFNαD, and chimeric IFNτ/IFNαD for seven days. Metabolic activity of HPBMC was assessed using WST-1 (Boehringer-Mannheim, Indianapolis, Ind.), which measures cell proliferation and viability based on the enzymatic activity of mitochondrial dehydrogenases in viable cells.

Following are examples which illustrate procedures for practicing the invention. These examples should not be construed as limiting. All percentages are by weight and all solvent mixture proportions are by volume unless otherwise noted.

### EXAMPLE 1

### IFNτ Inhibits Production of OVA-Specific IgE Antibody in Mice

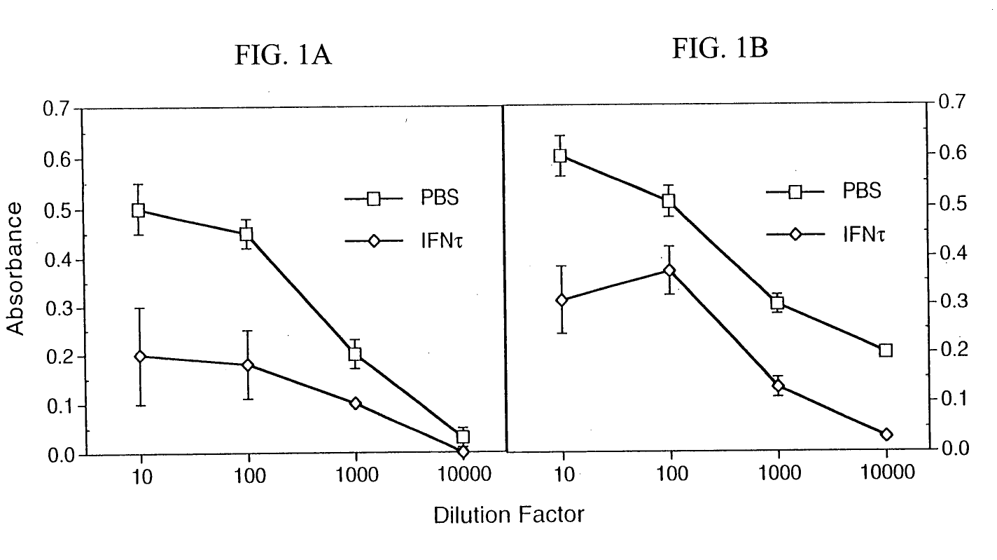

As shown in FIG. 1A, daily injections (ip) of INFτ at 5×10⁵ units beginning prior to OVA-aluminum hydroxide injection (ip) blocked IgE antibody production by over 50%. Further, the blocking continued even when the mice where challenged with aerosolized OVA (FIG. 1B). Thus, IFNτ inhibited OVA-specific IgE antibody production under conditions where the mice were immunized to OVA by injection and challenged by inhalation.

### EXAMPLE 2

### Reduced Inflammatory Cell Infiltration of IFNτ-Treated Mice

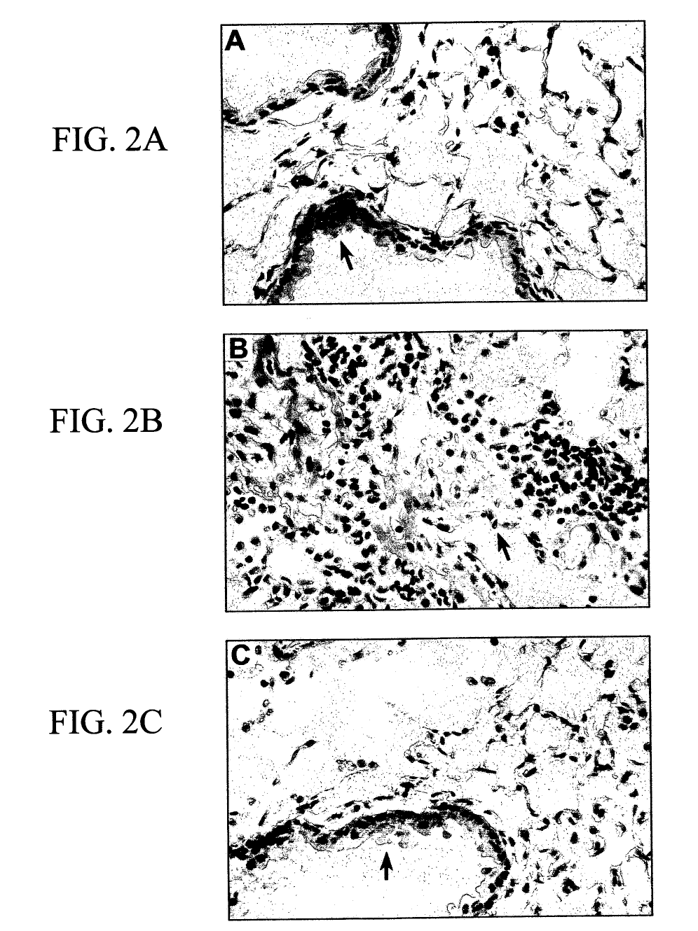

OVA immunized mice were challenged with aerosolized OVA following treatment with IFNτ in order to determine if the IFN treatment inhibited inflammatory cell infiltration into the lungs. IFNτ inhibition of cellular infiltration is shown in FIG. 2 where lung sections of naive (FIG. 2A), PBS treated (FIG. 2B), and INFτ treated (FIG. 2C) mice are compared. The destruction of the integrity of the epithelial tissue lining the bronchiole of the PBS treated mice (FIG. 2B) versus the protection in IFNτ treated mice (FIG. 2C) was evident. Eosinophil, basophil, and lymphocytic infiltration was assessed around bronchioles and blood vessels of INFτ and PBS treated (control) mice. Significantly fewer bronchioles had peribronchiolar aggregates of granulocytes with INFτ treatment (20%) as compared to PBS treatment (64%) (Table I).

TABLE I
IFNτ reduces OVA-induced inflammatory
cell infiltration into the bronchioles*

Lung sections

| | Granulocyte | Lymphocyte |
|---|---|---|
| | aggregates | aggregates |
| Treatment | (% Airways) | (% Airways) |
| PBS | 64 ± 6.0 | 78 ± 7.0 |
| IFNτ | 20 ± 7.0 | 34 ± 3.0 |
| Naive | 0 | 0 |

| | Blood smears | | | | |
|---|---|---|---|---|---|
| | Eosinophil | Basophil | Neutrophil | Monocyte | Lymphocyte |
| | (%) | (%) | (%) | (%) | (%) |
| PBS | 11 ± 5.0 | 6.0 ± 1.0 | 20 ± 0.7 | 10 ± 1.4 | 58 ± 6.0 |
| IFNτ | 2.5 ± 0.7 | 1.5 ± 0.7 | 15 ± 1.4 | 5.0 ± 1.4 | 72 ± 8.0 |
| Naive | 0.2 ± 0.3 | 0.5 ± 0.7 | 19 ± 1.4 | 10 ± 0.7 | 71 ± 4.0 |
| *Mice were treated (ip) with 5 × 10⁵ U of IFNτ daily starting three days prior to OVA immunization as described in the | | | | | |
| # Materials and Methods section. Airways having peribronchiolar aggregates were enumerated and divided by the total number of bronchioles | | | | | |
| # examined in each section. Differential white cell counts from blood smears are presented as percent cell type. Statistical significance | | | | | |
| # for the inhibition of granulocytes and lymphocyte aggregates around the bronchioles was shown by χ² test for IFNτ | | | | | |
| # treatment as compared to PBS treatment (p < 0.001). Statistical significance for the inhibition of eosinophil (p < 0.05) and | | | | | |
| # basophil (p < 0.1) by IFNτ treatment as compared to PBS treatment was shown by Student's t test. | | | | | |

Also, 78% of lung bronchioles in PBS-treated mice had lymphocytic infiltration compared to 34% for INFτ treatment. In addition, differential counts on the blood of mice showed that IFNτ-treated mice had lower levels of eosinophils and basophils as compared to those of PBS-treated groups, while neutrophil, monocyte, and lymphocyte levels were not significantly different from either the PBS-treated or naive (nonimmunized) mice. Thus, the data show that IFNτ treatment inhibits inflammatory cell infiltration into the lungs of OVA-sensitized- mice when exposed to aerosolized OVA allergen.

### EXAMPLE 3

### IFNτ-Treated Mice Have Lower IL-4 Levels than Control Mice

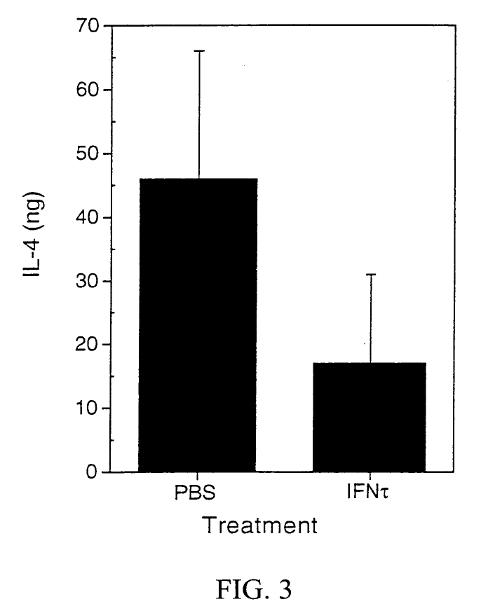

IL-4 levels in sera of mice treated with PBS, IFNτ, or nonimmunized (naive) mice were measured after aerosolized OVA exposure, which was given 20 days after immunization. It has been shown previously that IL-4 may be necessary for inducing the IgE isotype class switch in B cells (Lanzavecchia et al., 1984; Coffmann, Carty et al., 1986; Coffmann, Ohara et al., 1986; Rothman et al., 1988). As shown in FIG. 3, IL-4 levels in the IFNτ-treated group were less than half of those of the PBS-treated group.

### EXAMPLE 4

### In Vivo IFNτ-Treatment Inhibits OVA-Specific Splenocyte Proliferation

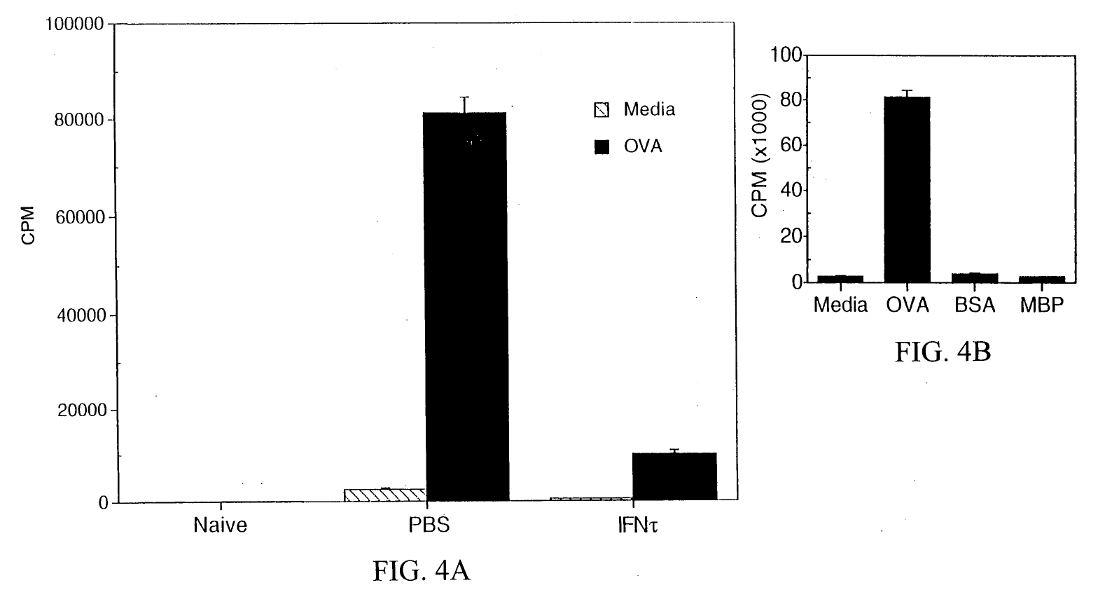

Spleens from nonimmunized (naive) mice and PBS- or IFNτ-treated OVA-immunized mice were removed 20 days after OVA immunization in order to determine the inhibitory effect of IFNτ treatment on OVA induced proliferation. Splenocytes were incubated in the presence of OVA for 84 h after which proliferation was assessed. As shown in FIG. 4, significantly reduced proliferation in response to OVA was observed in splenocytes from IFNτ-treated mice as compared to PBS-treated control mice. This proliferative activity was specific for OVA since bovine serum albumin (BSA) and myelin basic protein (MBP) did not activate splenocytes (FIG. 4 inset). Thus, in vivo IFNτ treatment of allergen-primed mice inhibited cellular proliferation in response to allergen.

### EXAMPLE 5

### In Vitro IFN Treatment of OVA-Sensitized Splenocytes Inhibits OVA-Specific Splenocytes Proliferation

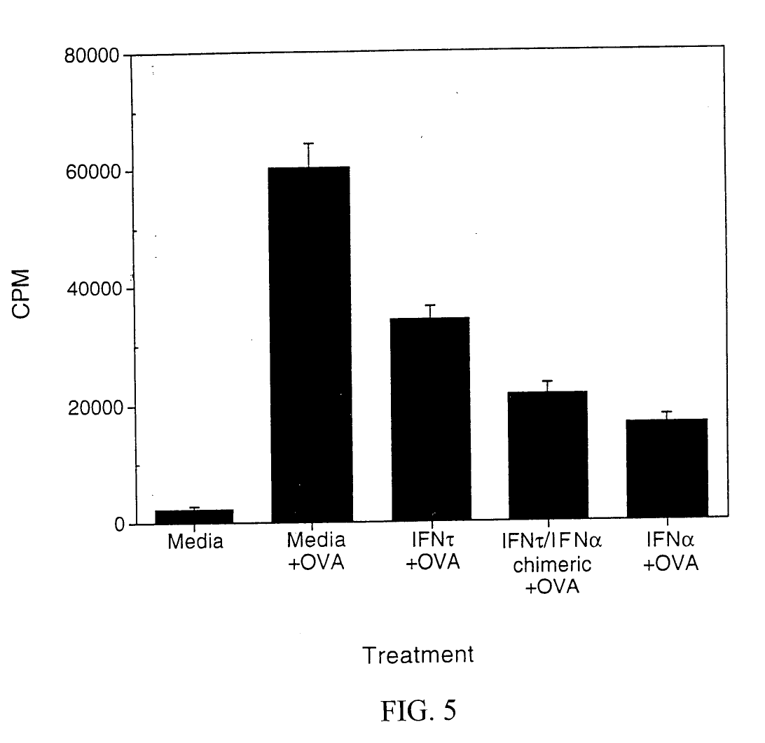

OVA sensitized splenocytes were treated in vitro with various IFNs in order to determine their effect on previously sensitized cells. Spleens were removed 20 days after OVA immunization and after aerosolized OVA treatment, and cultured with various type I IFNs for 84 h after which proliferation was assessed. In addition to ovine IFNτ treatment, human IFNαD, and chimeric IFNτ/IFNαD were also tested for their effects on OVA induced proliferation. The chimeric was tested as a potential for “humanized” IFNτ for possible human therapy. As shown in FIG. 5, both IFNτ and IFNαD inhibited OVA-specific splenocyte proliferation. Furthermore, the IFNτ/IFNαD chimeric, which contained amino acid residues 1-27 of ovine INFτ and residues 28-166 of human IFNαD, also had an inhibitory effect. Thus, treatment of OVA-sensitized splenocytes in vitro with type I IFNs suppressed cell proliferation.

### EXAMPLE 6

### Type I IFNs Inhibit Mouse and Human IgE Production

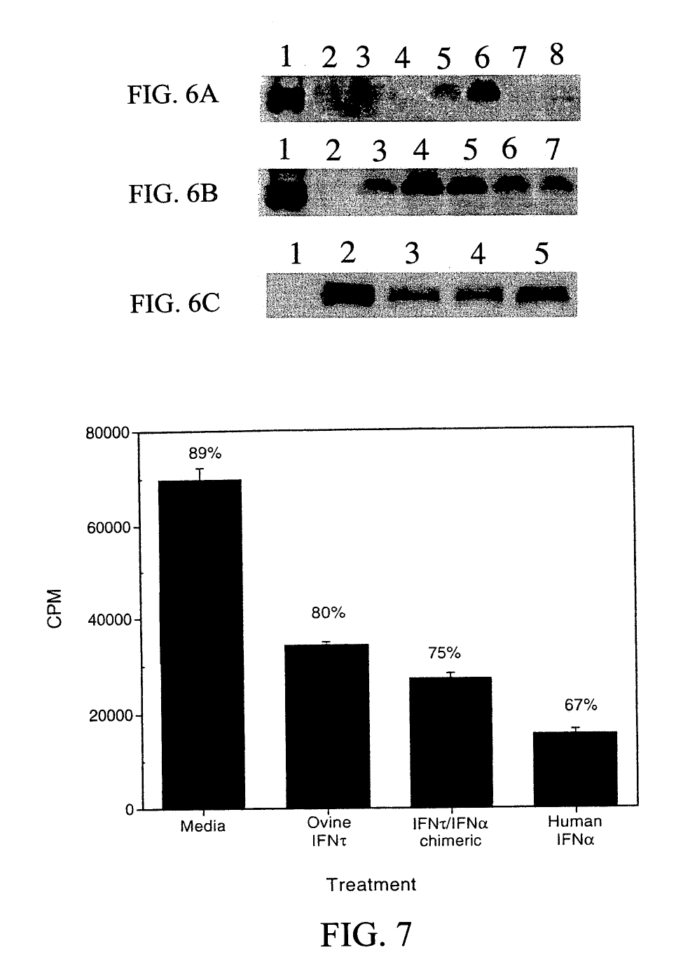

Immunoblots for the detection of IgE antibodies were performed on culture supernatants taken from the proliferation assay experiments performed in FIGS. 4 and 5. As shown in FIG. 6A, IgE was detected in cultures containing PBS-treated splenocytes that were incubated in the presence of OVA. There was little or no IgE in supernatants from splenocytes of IFNτ-treated mice. Immunoblots for detection of IgE in culture supernatants from in vitro IFN treatment of OVA sensitized mouse splenocytes showed an inhibition of IgE production by all of the type I IFNs as compared to the media control (FIG. 6B). The chimeric IFNτ/IFNαD protein and the IFNαD protein were better inhibitors than was IFNτ, however.

The U266BL human myeloma cell line, which produces IgE antibodies constitutively was also incubated with the type I IFNs in order to determine if the IFNs had a direct effect on the human IgE-producing B cells. Cells were starved overnight prior to treatment with various type I IFNs, including the IFNτ/IFNαD chimeric. After incubation with the IFNs for 96 h, supernatants were collected and IgE levels were detected by immunoblot. As shown in FIG. 6C, IgE levels were lower in the IFN treated groups as compared to the media control. Thus, type I IFNs inhibit U266BL human myeloma cells and OVA-specific mouse B cells from producing IgE antibodies.

### EXAMPLE 7

### IFN Inhibition of Proliferation of the Human IgE-Producing Myeloma Cells

The U266BL myeloma cells were cultured in the presence of 10,000 U/ml of IFNs for 72 h, after which proliferation was measured. All IFNs inhibited proliferation of the cells by about 50% or more with IFNαD being the most effective and INFτ being the least effective (FIG. 7). Viabilities were determined and showed that the IFNαD was the most toxic (67% viability) as compared to the INFτ (80% viability) and the IFNτ/IFNα chimeric (75% viability). The IFNτ/IFNαD chimeric suppressed proliferation more effectively than IFNτ but not as effectively as IFNαD. Thus, type I IFNs, with various toxicity levels, inhibit the cell proliferation of the human IgE producing cell line, U266BL.

### EXAMPLE 8

### Lack of Toxicity of the IFNτ/IFNαD Chimeric on Human Peripheral Blood Mononuclear Cells (HPBMC)

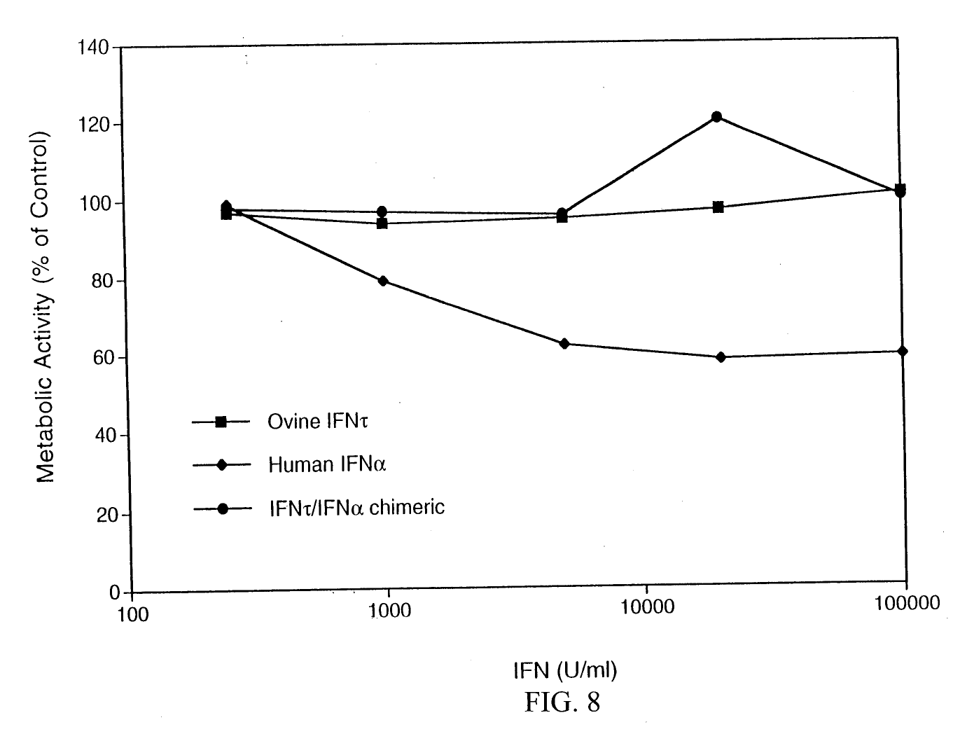

The IFNτ/IFNαD chimeric was compared with recombinant ovine INFτ and recombinant human IFNαD for toxicity on HPBMC. After seven days of treatment of the HPBMC with various concentrations of IFNs (250 to 100,000 U/ml), toxicities were measured based on the enzymatic activity of the mitochondrial dehydrogenases in viable cells. As shown in FIG. 8, human IFNαD was toxic at concentrations of 1,000 to 100,000 U/ml as compared to ovine INFτ and IFN/IFNαD chimeric, which did not show toxicity at any concentration. Thus, the IFNτ/IFNαD chimeric, like ovine IFNτ, lacked the toxicity associated with human IFNαD.

It should be understood that the examples and embodiments described herein are for illustrative purposes only and that various modifications or changes in light thereof will be suggested to persons skilled in the art and are to be included within the spirit and purview of this application and the scope of the appended claims.

### References

Bazer, F. W., H. L. Johnson, J. K. Yamamoto (1989) “Effect of ovine trophoblast protein-1 (oTP-1) on replication of feline (FIV) and human (HIV) immunodeficiency viruses (abstr)” Biol. Reprod. 40 (Suppl.):63.

Bazer, F. W ., H. M. Johnson (1991) “Type I conceptus interferons: Maternal recognition of pregnancy signals and potential therapeutic agents” Am. J Reprod. Immunol. 26:19-22.

Beck L., H. L. Spiegelberg (1989) “The polyclonal and antigen-specific IgE and IgG subclass response of mice injected with ovalbumin in alum or complete freund's adjuvant” Cellular Immunol. 123:1-8.

Benoit, P., D. Maguire, I. Plavec, H. Kocher, M. Tovey, F. Meyer (1993) “A monoclonal antibody to recombinant human IFN-alpha receptor inhibits biological activity of several species of human IFN-alpha, IFN-beta, and IFN-omega. Detection of heterogeneity of the cellular type I IFN receptor” J. Immunol. 150(3):707-716.

Bonnem, E. M., R. J. Spiegel (1984) “Interferon-alpha: current status and future promise” J. Bio. Response Modifiers 3(6):580-598.

Coffman R. L., J. Carty (1986) “A T cell activity that enhances polyclonal IgE production and its inhibition by Interferon-γ” J Immunol. 136:949-954.

Coffman, R. L., J. Ohara, M. W. Bond, J. Carty, A. Zlotnik, W. E. Paul (1986) “B cell stimulatory factor-1 enhances the IgE response of lipopolysaccharide-activated B cells” J. Immunol. 136:4538-4541.

Davis, G. L., L. A. Balart, E. R. Schiff et al. (1989) “Treatment of chronic hepatitis C with recombinant interferon alfa. A multicenter randomized, controlled trial” N. Engl. J. Med. 321(22):1501-1506.

DeMaeyer, E. et al. (1988) Interferons and Other Regulatory Cytokines John Wiley and Sons, New York.

Dolen, J. G., A. Mathur (1995) “Undetectable interferon-alpha serum levels in a patient with atopic dermatitis” J. Interferon Cytokine Res. 15(11):973-975.

Dusheiko, G. M., A. C. Paterson, L. Pitcher et al. (1986) “Recombinant leucocyte interferon treatment of chronic hepatitis B. An analysis of two therapeutic trials” J. Hematology 3(suppl.2):S 199-S207.

Finter, N. B., S. Chapman, P. Dowd et al. (1991) “The use of interferon-alpha in virus infections” Drugs 42(5):749-765.

Godkin, J. D., F. W. Bazer, J. Moffatt, F. Sessions, R. M. Roberts (1982) “Purification and properties of a major, low molecular weight protein released by the trophoblast of sheep blastocysts at day 13-21” J. Reprod. Fertil. 65:141-150.

Gruschwitz, M. S., K. P. Peters, A. Heese, N. Stosiek, H. U. Koch, O. P. Hornstein (1993) “Effects of interferon-alpha-2b on the clinical course, inflammatory skin infiltrates and peripheral blood lymphocytes in patients with severe atopic eczema” Int. Arch Allergy Immunol. 101(1):20-30.

Hamelmann, E., A. Oshiba, J. Paluh, K. Bradley, J. Loader, T. A. Potter et al. (1996) “Requirement for CD8⁺ T cells in the development of airway hyperresponsiveness in a murine model of airway sensitization” J. Exp. Med. 183:1719-29.

Hamelmann, E., A. Oshiba, J. Schwarze, K. Bradley, J. Loader, G. L. Larsen et al. (1997) “Allergen-specific IgE and IL-5 are essential for the development of airway hyperresponsiveness” Am. J. Respir. Cell Mol. Biol. 16:674-82.

Heeke, G. V., T. L. Ott, A. Strauss, D. Ammaturo, F. W. Bazer (1996) “High yield expression and secretion of the ovine pregnancy recognition hormone interferon-tau by Pichia pastoris” J. Interferon Res. 16:119-126.

Hofstra, C. L., I. Van Ark, G. Hofman, F. P. Nijkamp, P. M. Jardieu, A. J. Van Oosterhout (1998) “Differential effects of endogenous and expgenous interferon-gamma on immunoglobulin E, cellular infiltration, and airway responsiveness in a murine model of allergic asthma” Am. J. Respir. Cell Mol. Biol. 19(5):826-835.

Imakawa, K., R. V. Anthony, M. Kazemi, K. R. Marotti, H. G. Polites, R. M. Roberts (1987) “Interferon-like sequence of ovine trophoblast protein secreted by embryonic trophectoderm” Nature 330:377-379.

Johnson, H. M., F. W. Bazer, B. E. Szente, M. A. Jarpe (1994) “How interferons fight disease” Sci. Am. 270(5):68-75.

Kay, A. B., C. J. Corrigan (1992) “Eosinophils and neutrophils” Br. Med. Bull. 48:51-64.

Khan, O. A., H. Jiang, P. S. Subramaniam, H. M. Johnson, S. S. Dhib-Jalbut (1998) “Immunomodulating functions of recombinant ovine interferon tau: potential for therapy in multiple sclerosis and autoimmune disorders” Multiple Sclerosis 4:63-69.

Kashima, H., B. Leventhal, K. Clark et al.(1988) “Interferon alfa-n1 (Wellferon) in juvenile onset recurrent respiratory papillomatosis: results of a randomized study in twelve collaborative institutions” Laryngoscope 98(3):334-340.

Kimata H., Y. Akiyama, M. Kubota, K. Furusho (1995) “Interferon-alpha treatment for severe atopic dermatitis” Allergy 50(10):837-840.

Lack, G., K. L. Bradley, E. Hamelmann, H. Renz, J. Loader, D. Y. Leung, G. Larsen, E. W. Gelfand (1996) “Nebulized IFN-gamma inhibits the development of secondary allergic responses in mice” J. Immunol. 157(4):1432-1439.

Lanzavecchia, A., B. Parodi (1984) “In vitro stimulation of IgE production at a single precursor level by human alloreactive T-helper clones” Clin. Exp. Immunol. 55:197-203.

Mancino, D., Z. Ovary (1980) “Adujuvant effects of amorphous silica and of aluminium hydroxide on IgE and IgG1 antibody production in different inbred mouse strains” Int. Archs Allergy appl. Immun. 61:253-258.

Miguel, N. M. R., E. M. Vaz (1977) “Effect of aluminium hydroxide gel on the Secondary response in mice” Int. Archs Allergy appl. Immun. 54:382-384.

Mujtaba, M. G., W. J. Steit, H. M. Johnson (1998) “IFNτ suppresses both the autoreactive humoral and cellular immune responses and induces stable remission in mice with chronic experimental allergic enephalomyelitis” Cellular Immunol. 186:94-102.

Mujtaba, M. G., J. M. Soos, H. M. Johnson (1997) “CD4 T suppressor cells mediate interferon tau protection against experimental allergic encephalomyelitis” J. Neuroimmunol. 75:35-42.

Nilsson, K., H. Bennich, S. G. O. Johansson, J. Ponten (1970) “Established immunoglobulin producing myeloma (IgE) and lymphoblastoid (IgG) cell lines from an IgE myeloma patient” Clin. Exp. Immunol. 7:477-489.

Noh, G. W., K. Y. Lee (1998) “Blood eosinophils and serum IgE as predictors for prognosis of interferon-gamma therapy in atopic dermatitis” Allergy 53(12): 1202-1207.

Oldham, R. K. (1985) “Biologicals for cancer treatment: interferons” Hospital Practice 20(12):71-91.

Pontzer, C. H., F. W. Bazer, H. M. Johnson (1991) “Antiproliferative activity of a pregnancy recognition hormone, ovine trophoblast protein-1” Cancer Res. 51:5304-5307.

Pontzer, C. H., T. L. Ott, F. W. Bazer, H. M. Johnson (1994) “Structure/function studies with interferon-tau: evidence for multiple active sites” J. Interferon Res 14:133-141.

Quesada, J. R., J. Reuben, J. T. Manning, E. M. Hersh, J. U. Gutterman (1984) “Alpha Interferon for Induction of Remission in Hairy-Cell Leukemia” New England Journal of Medicine 310:15-18.

Revoltella, R., Z. Ovary (1969) “Preferential production of rabbit reaginic antibodies” Int Arch Allergy 36:282-289.

Rothman, P., S. Lutzker, W. Cook, R. Coffman, F. W. Alt (1988) “Mitogen plus interleukin-4 induction of Cε transcripts in B-lymphoid cells” J. Exp. Med. 168:2385-9.

Soos, J. M., H. M. Johnson (1995) “Type I interferon inhibition of superantigen-induced stimulation: Inplications for the treatment of superantigen-associated disease” J. IFN Cytokine Res. 15:39-45.

Soos, J. M., P. S. Subramaniam, A. C. Hobeika, J. Schiffenbauer, H. M. Johnson (1995) “The interferon pregnancy recognition hormone, interferon tau, blocks both development and superantigen reactivation of experimental allergic encephalomyelitis without associated toxicity” J. Immunol. 155:2747-2753.

Soos, J. M., M. G. Mujtaba, P. S. Subramaniam, W. J. Steit, H. M. Johnson (1997) “Oral feeding of interferon τ can prevent the acute and chronic relapsing forms of experimental allergic encephalomyelitis” J. Neuroimmunol. 75:43-50.

Subramaniam, P. S., S. A. Khan, C. H. Pontzer, H. M. Johnson (1995) “Differential recognition of the type I interferon receptor by interferon τ and α is responsible for their disparate cytotoxicities” Proc. Natl. Acad. Sci. USA 92:12270-12274.

Warner, N. L., N. M. Vaz, Z. Ovary (1968) “Immunoglobulin classes in antibody responses in mice. I. Analysis by biological properties” Immunology, Long 14:725-34.

Wei, C. F. et al. (1983) “Isolation and comparison of two molecular species of the BAL 31 nuclease from Alteromonas espejiana with distinct kinetic properties” J. Biol. Chem. 258(22):13506-13512.

Whaley, A. E., C. S. Meka, L. A. Harbison, J. S. Hunt, K. Imakawa (1994) “Identification and cellular localization of unique interferon mRNA from human placenta” J. Biol. Chem. 269:10864-10868.

We claim:

1. A method for suppressing or inhibiting IgE production, said method comprising administering an effective amount of interferon tau or a chimeric interferon, wherein said chimeric interferon comprises a mammalian interferon tau amino terminus and a human type I interferon carboxy terminus other than interferon tau, or a biologically active fragment of said interferon tau or said chimeric interferon.

2. The method according to claim 1, wherein said mammalian interferon tau amino terminus is from a mammal selected from the group consisting of primate, ovine and bovine.

3. The method according to claim 1, wherein said chimeric interferon comprises amino acid residues from about amino acid residue 1 to about amino acid residue 27 of ovine interferon tau and amino acid residues from about amino acid residue 28 to about amino acid residue 166 of human interferon alpha.

4. The method according to claim 3, wherein said interferon alpha is interferon alpha D.

5. The method according to claim 1, wherein said interferon tau or said chimeric interferon is administered to a person or animal in need of suppression or inhibition of IgE production.

6. The method according to claim 1, wherein said suppression or inhibition of IgE production occurs through inhibition of B-cell IgE secretion or inhibition of B-cell proliferation.

7. The method according to claim 5, wherein said interferon tau or said chimeric interferon is administered by routes selected from the group consisting of oral administration, parenteral administration, subcutaneous administration and intravenous administration.

8. The method according to claim 7, wherein said person or animal is afflicted with, or predisposed to, an IgE-related condition.

9. The method according to claim 8, wherein said allergic condition is selected from the group consisting of allergic rhinitis, atopic dermatitis, bronchial asthma and food allergy.

10. The method according to claim 1, wherein said interferon tau or said chimeric interferon is administered in vitro.

11. The method according to claim 1, wherein said interferon tau or said chimeric interferon is formulated in a pharmaceutically acceptable carrier or diluent.

12. A composition comprising a chimeric type I interferon, or a biologically active mutein, fragment, variant or peptide thereof, which is capable of suppressing or inhibiting IgE production, wherein said chimeric IFN comprises part of at least two IFNs selected from the group consisting of IFNα, IFNβ, IFNτ and IFNω.

13. The composition according to claim 12, wherein said suppression or inhibition of IgE production occurs through inhibition of B-cell IgE secretion or inhibition of B-cell proliferation.

14. The composition according to claim 12, wherein said chimeric IFN comprises a mammalian IFNτ amino terminus and a human type I IFN carboxy terminus other than IFNτ.

15. The composition according to claim 14, wherein said mammalian IFNτ amino terminus is from a mammal selected from the group consisting of primate, ovine and bovine.

16. The composition according to claim 14, wherein said chimeric IFN comprises amino acid residues from about 1 to about 27 of ovine INFτ and amino acid residues from about 28 to about 166 of human IFNα.

17. The composition according to claim 16, wherein said IFNα is IFNαD.

18. The composition according to claim 12, wherein said chimeric IFN is recombinantly produced and is expressed in Pichia pastoris.

19. A method for suppressing or inhibiting IL-4 production, said method comprising contacting an immune cell with a type I interferon, or a biologically active mutein, fragment, variant or peptide thereof.
